(19) United States
(12) Patent Application Publication
(10) Pub. No.: US 2004/0047922 A1
(43) Pub. Date: Mar. 11, 2004

(54) Preparation with vascular protective and anti-oxidative effect and use thereof

(76) Inventor: Erich Elstner, Grobenzell (DE)

Correspondence Address: Richard L Byrne, 436 Seventh Avenue, 700 Koppers Building, Pittsburgh, PA, 15219-1818, US

(21) Appl. No.: 10/311,730

(22) PCT Filed: May 28, 2001
(86) PCT No.: PCT/DE01/02082

(30) Foreign Application Priority Data
Jul. 28, 2000 (DE) 10038640.7

(51) Int. Cl. A61K 38/43; A61K 31/355; A61K 35/78; A61K 31/01

(52) U.S. Cl. 424/736; 514/458; 514/762; 424/94.1

(57) ABSTRACT

A new preparation is provided that has a vasculoprotective effect and helps prevent the development of atherosclerosis. The preparation comprises a terpinene-containing etherial oil or terpinene.

This invention relates to a preparation with a vasculoprotective and antioxidant effect and its use.

Hypercholesterolamia, in particular, an increased cholesterol-carrying fraction (low density lipoprotein, LDL) and the oxidative change of LDL in the blood and in the endothelial lesions of arterial vessels are major factors that cause the development of atherosclerosis. Atherosclerosis is an insidious disease that develops over decades by lipid deposits in the human arterial system.

This disease can result in an occlusion of the coronary arteries (cardiac infarction) due to growing plaques (lipid deposits inside the vessels) or lesions. In addition, plaque that is washed off may cause an occlusion of an artery in the brain (stroke). But atherosclerosis also develops in other parts of the circulatory system.

A drastic inhibition of LDL oxidation (LDL oxidation is most important in atherosclerosis development from a quantitative point of view) using antioxidants can reduce the risks of suffering a cardiac infarction or a stroke.

The development of atherosclerosis is mainly due to oxidation of so-called lipoproteins, particularly of LDL. Lipoproteins are macromolecular complexes of protein and lipid that are characterized by physical and chemical parameters such as salt density and ultracentrifugation as well as by special proteins (apolipoproteins). The lipoproteins circulate in the blood and enable transport and transfer of water-insoluble fats such as cholesterol, neutral fat (triglycerides) and phospholipids; depending. on their hydrated density a distinction is made between very low density lipoproteins (VLDL), low density lipoproteins (LDL), and high density lipoproteins (HDL). Increased levels of LDL cholesterol and its oxidative state are mainly responsible for the development of atherosclerosis.

LDL is the main carrier molecule for cholesterol and cholesterol ester in the blood plasma. It consists of a lipid core that is surrounded by a shell of phospholipids and unesterified cholesterol. A protein molecule (apo B-100) is embedded in this shell.

The LDL cholesterol is partly biologically oxidized in the blood plasma, which is possible due to activated oxygen species under radical formation. Additional oxidation takes place in the atherosclerotic plaque by endothelial cells (the inside coat of an artery, also called tunica intima) and by unstriped muscle cells (the middle coat of an artery, media). Lipid peroxidation processes in the LDL decompose the polyunsaturated fatty acids of LDL into various products. These processes yield, inter alia, reactive aldehydes that react with the amino acids of apo B-100 and cause further modification. LDL modification, i. e. oxidative change of its fat and protein portions, causes a recognition problem by an important endogenic receptor system whose function it is to metabolize LDL cholesterol in various tissues of the human body. These modified LDL that are not recognized by their actual are absorbed by macrophages (four different receptors for absorbing oxidized LDL are known as yet) in an uncontrolled way and deposited in the tunica intima. This results in a dysfunction of this section (endothelium, tunica intima, and unstriped muscle cells) of the arterial wall and the formation of plaques or vascular lesions, the initial phase of atherosclerosis.

It has been proven in many animal models using various antioxidants that these agents inhibit LDL, VLDL, and HDL oxidation and therefore prevent the development of atherosclerosis, various ingredients of plant extracts have been studied in this way. These are mostly aqueous extracts that contain flavonoids.

It is therefore the object of this invention to provide a new preparation that prevents LDL oxidation in the blood plasma.

This problem is solved by the preparation that has the characteristics listed in claim 1.

According to the invention, this preparation contains a terpinene-containing etherial oil or terpinene. Preferred are etherial oils from citrus fruit, in particular, lemons, that contain γ-terpinene as a natural ingredient.

When using etherial oils for producing the preparation according to the invention, it may also be used in a terpinene-enriched form.

According to a particularly preferred embodiment of the invention, the preparation additionally contains α-tocopherol (vitamin E) and/or coenzyme Q (Q₁₀). The combination according to the invention with these active ingredients results in an advantageous synergetic effect that a person skilled in the art could not have predicted. The result is a distinct inhibition of LDL oxidation in the blood.

In-vitro LDL oxidation is a common model for testing various substances for their antioxidant properties because they accumulate in the LDL in vitro due to pre-incubation of the plasma with (primarily lipophilic) test substances (McLean und Hagaman, 1989, Biochemistry 28(1); pp. 321-327, Esterbauer et al, 1991b, Am. J. Clin. Nutr. 53, pp. 315S-321S). After accumulation, the influence of these substances on the oxidability of LDL can be studied.

An antioxidant and thus lipid-reducing effect of lemon oil or γ-terpinene was detected in the LDL oxidation model. This effect may be due to structural characteristics: a relatively stable tertiary radical can be formed on the isopropyl group by H-abstraction of fatty acid peroxyl radicals, and this radical is in addition stabilized by a resonating double bond.

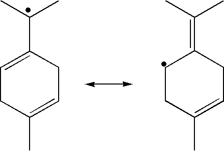

The protection of the LDL from oxidation by lemon oil or the γ-terpinene it contains is thus based on the capability of this oil to react with the lipid peroxyl radicals and in this way to interrupt the chain reaction of lipid peroxidation and to delay protein oxidation.

The preparation of the invention can be used in various areas. Thus the preparation can be used as a drug, a nutritional supplement and/or dietetic product, and other active ingredients, harmless additives and/or adjuvants can be contained as required.

According to a preferred embodiment of the preparation of the invention, the following concentrations of ingredients are used:

| | |
|---|---|
| γ-terpinene | 0.5-20 percent by weight |
| α-tocopherol | 10-50 percent by weight |
| coenzyme Q (Q₁₀) | 10-50 percent by weight |

Other advantageous embodiments are described in the dependent claims.

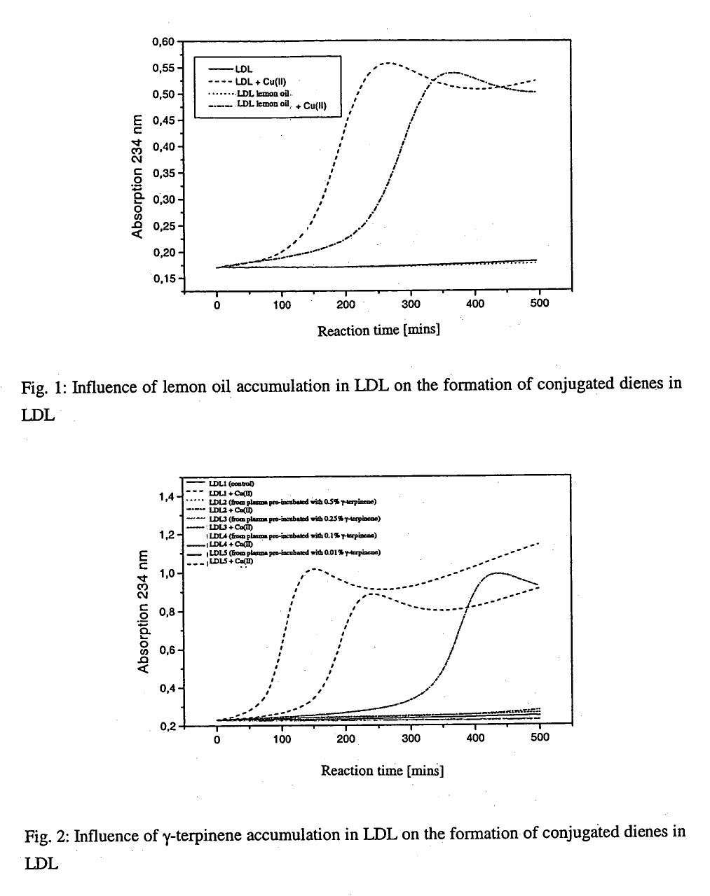

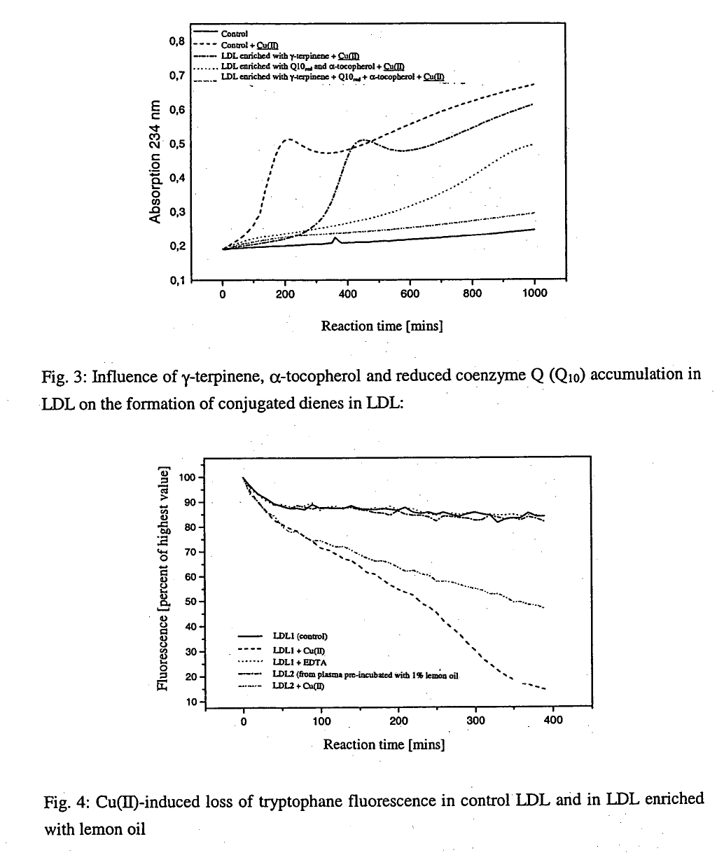

The invention shall be explained in more detail below with reference to findings that are also shown in FIGS. 1 to 5. Wherein:

Abb. 1 Influence of lemon oil accumulation in LDL on the formation of conjugated dienes in LDL

Abb. 2 Influence of γ-terpinene accumulation in LDL on the formation of conjugated dienes in LDL

Abb. 3 Delay in the formation of conjugated dienes in LDL due to accumulation of γ-terpinene, α-tocopherol and reduced coenzyme Q (Q₁₀) therein: an unexpected effect,

Abb. 4 Cu(II)-induced loss of tryptophane fluorescence in control LDL and in LDL enriched with lemon oil, and

Abb. 5 Cu(II)-induced loss of tryptophane fluorescence in control LDL and in LDL enriched with γ-terpinene.

The Influence of Lemon Oil and γ-Terpinene on the Formation of Conjugated Dienes in LDL

Continuous measurement of the formation of conjugated dienes in LDL is an accepted method of comparing oxidizability of the lipid portion of various LDL samples (Esterbauer et al. 1989, Free Rad. Res. Comms. 6(1), pp. 67-75; Parthasarathy et al. 1998, Free Rad. Res. 28, pp. 583-591). The lag phase shows the oxidizability of LDL; a longer lag phase means greater resistance to oxidation. The study was to find out whether accumulation of lemon oil or γ-terpinene in LDL can protect the LDL from Cu(II)-induced oxidation. As FIG. 1 shows, accumulation of lemon oil clearly extends the formation of conjugated dienes in LDL.

In another test, blood plasma was incubated with 0.5, 0.25, 0.1 and 0.01% γ-terpinene and the LDL isolated from it was tested for resistance against cooper-induced oxidation. It was found that the lag phase expands depending on concentration. The lag phase is clearly extended with 0.01% γ-terpinene in the plasma, 0.1% γ-terpinene in the plasma extends the lag phase by about 250 minutes the samples with higher concentrations in the plasma did not even reach the propagation phase after 500 minutes (FIG. 2).

The resistance of LDL to oxidation can be considerably increased once more when incubating plasma with γ-terpinene and adding α-tocopherol and coenzyme Q.

The Influence of Lemon Oil and γ-Terpinene on the Cu(II)-Induced Loss of Tryptophane Fluorescence in LDL

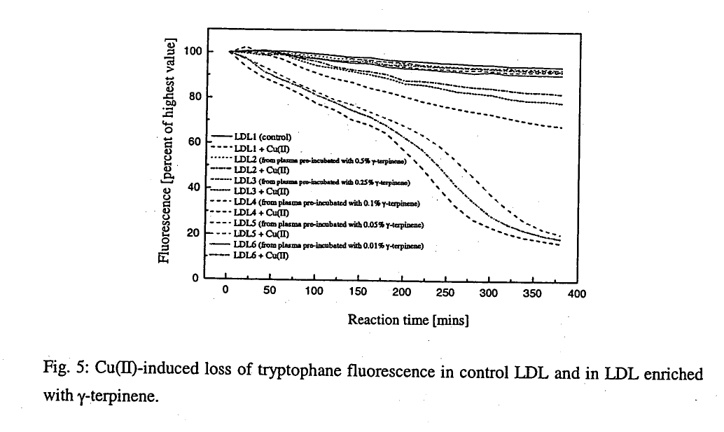

LDL shows fluorescence in the UV range due to the 37 tryptophane residues in apo B-100. Oxidation of HDL or LDL with Cu(II) goes along with a reduction of the tryptophane residues (Reyftmann et al., 1990, Biochim. Biophys. Acta 1042, pp. 159-167), which can be observed by measuring fluorescence. After adding Cu(II) to an LDL solution, fluorescence initially drops within a few seconds due to quenching effects caused by copper. Then fluorescence shows a more or less linear decline, and in a second phase fluorescence diminishes rapidly. The time before the slower phase changes into the faster phase can be defined as the lag phase, just like with diene conjugation. (Giessauf et.al., 1995, Biochim. Biophys. Acta 1256, pp. 221-232). The faster decline in fluorescence starts about at the same time as the propagation phase of diene conjugation and is most likely based on a reaction of lipid peroxidation products with tryptophane residues. We used this test system, too, to find out whether the accumulation of lemon oil or γ-terpinene in LDL influences the Cu(II)-induced loss of tryptophane fluorescence. It is apparent that lemon oil can considerably delay late protein oxidation and that γ-terpinene also slows down early protein oxidation; a concentration of 0.5% of γ-terpinene in the plasma almost completely prevents oxidation of tryptophane residues in LDL (FIG. 4 and FIG. 5).

The preparation according to the invention can be processed into various forms of administration. It may be used as a drug, a nutritional supplement, or a dietetic product. For example, it can be diluted to be administered as a syrup or in drops. It may also be added to liquids such as milk serum or to solids such as roughage or cereals.

The preparation according to the invention may additionally contain harmless natural or synthetic additives or adjuvants such as binding agents, blasting agents, lubricating agents, separating agents, solvents, stabilizers, dyes and flavor corrigents, as the form of administration may allow. Examples of adjuvants that can be used according to the invention are

binding agents such as starch, alginate, gelatin, sugar, carob seed flour, cellulose derivatives such as cellulose ether, and polymers such as polyvinyl pyrrolidone;

blasting agents such as starch and hydroxyethyl starch;

lubricating and separating agents such as talc, stearates such as calcium and magnesium stearate, magnesium and calcium carbonate, cellulose, magnesium oxide, colloidal silica gel, silicates such as sodium, magnesium, calcium and aluminum silicate, separating flours such as bread flour, spelt flour, potato flour, buckwheat flour, wood flour and carob seed flour;

solvents such as water, alcohol and solutions of binding agents;

stabilizers such as fats, oils, flavoring agents, and starch derivatives; Coloring agents such as natural and synthetic dyes and pigments approved under legislation relating to food and drugs such as carotene, sugar coloring, betanine and lycopine; and

flavor corrigents such as spices, salts, sweeteners, and flavoring agents.

The adjuvants listed above are particularly suitable for producing tablets or granulate. When used as a nutritional supplement, the preparation according to the invention can be added to the desired product at any stage of production.

1. A preparation with a vasculoprotective and antioxidant effect, characterized in that it contains a terpinene-containing etherial oil or terpinene.

2. The preparation according to claim 1 wherein the terpinene is γ-terpinene.

3. The preparation according to claim 1 or 2 wherein the etherial oil is lemon oil.

4. The preparation according to any one of claims 1 through 3, characterized in that it contains other active ingredients, harmless additives and/or adjuvants.

5. The preparation according to claim 4, characterized in that it contains α-tocopherol.

6. The preparation according to any one of claims 4 or 5, characterized in that it contains coenzyme Q (Q₁₀).

7. The preparation according to claim 1 wherein the etherial oil is enriched with terpinene.

8. Use of the preparation according to claims 1 through 7 as a drug.

9. Use of the preparation according to claims 1 through 7 as a nutritional supplement.

10. Use of the preparation according to claims 1 through 7 as a dietetic product.

12. The preparation according to claim 11, wherein the terpinene is γ-terpinene.

13. The preparation according to claim 11, wherein the etherial oil is lemon oil.

14. The preparation according to claim 11, wherein the preparation contains other active ingredients, harmless additives and/or adjuvants.

15. The preparation according to claim 14, wherein the preparation contains α-tocopherol.

16. The preparation according to claim 14, wherein the preparation contains coenzyme Q (Q₁₀).

17. The preparation according to claim 11, wherein the etherial oil is enriched with terpinene.

18. The use of the preparation according to claim 11 as a drug.

19. The use of the preparation according to claim 11 as a nutritional supplement.

20. The use of the preparation according to claim 11 as a dietetic product.

21. A preparation having a vasculoprotective and antioxidant effect comprising a terpinene material selected from γ-terpinene, lemon oil and mixtures thereof, α-tocopherol and coenzyme Q (Q₁₀).

22. The preparation of claim 21 processed into a form for administration as a drug.

23. The preparation of claim 21 processed into a form for administration as a nutritional supplement.

24. The preparation of claim 21 processed into a form for administration as a dietetic product.
